(19) United States
(12) Patent Application Publication
(10) Pub. No.: US 2025/0222438 A1
(43) Pub. Date: Jul. 10, 2025

(54) FISCHER-TROPSCH SYNTHESIS CATALYST, PREPARATION METHOD THEREFOR, AND APPLICATION THEREOF

(71) Applicants: CHINA ENERGY INVESTMENT CORPORATION LIMITED, Beijing (CN); NATIONAL INSTITUTE OF CLEAN-AND-LOW-CARBON ENERGY, Beijing (CN)

(72) Inventors: Shaodong QIN, Beijing (CN); Jiabo LI, Beijing (CN); Ruonan HE, Beijing (CN); Bingfeng YAN, Beijing (CN)

(21) Appl. No.: 18/700,335

(22) PCT Filed: Nov. 23, 2021
(86) PCT No.: PCT/CN2021/132444

§ 371 Date: Apr. 11, 2024

(30) Foreign Application Priority Data
Oct. 11, 2021 (CN) 202111180725.X

(51) Int. Cl. B01J 27/135; B01J 21/06; B01J 35/30; B01J 37/04; B01J 37/08; C07C 1/04

(52) U.S. Cl. CPC B01J 27/135; B01J 21/063; B01J 35/393; B01J 37/04; B01J 37/088; C07C 1/043; C07C 2521/06; C07C 2523/75

(57) ABSTRACT

A Fischer-Tropsch synthesis catalyst, a preparation method thereof, and an application thereof, relating to the field of Fischer-Tropsch synthesis catalysts. The catalyst comprises: 10 wt % to 45 wt % of Co, 0.01 wt % to 2.5 wt % of Mn, 0.01 wt % to 1.5 wt % of Cl, 0.5 wt % to 8 wt % of ZrO₂, and 35 wt % to 85 wt % of carrier TiO₂; a molar ratio of Cl to Zr is 1:20 to 1:0.1; and the particle size of cobaltosic oxide in the catalyst ranges from 16 nm to 27 nm. The TiO₂ is composed of anatase and rutile crystal forms, and the content of the anatase is richer than the content of rutile. The prepared catalyst has low methane selectivity, high activity, good sintering resistance and hydrothermal resistance, and stability.

### CROSS-REFERENCE TO RELATED APPLICATIONS

The present application claims priority to Chinese Patent Application 202111180725.X filed on Oct. 11, 2021, and the entire disclosure of which is incorporated herein by reference.

### TECHNICAL FIELD

The present invention relates to the field of Fischer-Tropsch synthesis catalysts, and particularly to a Fischer-Tropsch synthesis catalyst, a preparation method thereof, and an application thereof.

### BACKGROUND OF THE INVENTION

The Fischer-Tropsch synthesis reaction is a process of converting a synthesis gas into hydrocarbon mixture through catalysts, and a reaction equation is as follows:

nCO+(2n+1)H₂→CₙH₂ₙ+2+nH₂O ΔH=−165KJ/mol

Fischer-Tropsch synthesis is the key technology of an indirect coal-to-oil and natural gas-to-oil technology, and the performance of the Fischer-Tropsch synthesis catalyst directly determines the economy and competitiveness of the whole indirect coal-to-oil and natural gas-to-oil technology. Commonly used Fischer-Tropsch synthesis catalysts comprise iron-based and cobalt-based types. Compared with the iron-based catalyst, the cobalt-based catalyst has obvious advantages of high Fischer-Tropsch synthesis activity and low CO₂ selectivity, thus being more extensively concerned and applied worldwide.

In the Fischer-Tropsch synthesis, besides the hydrocarbon mixture, it also generates a large amount of steam. So that, in industrial production process, especially tubular fixed bed reactor, there are severe requirements for the hydrothermal stability of the cobalt-based catalyst.

In addition, the Fischer-Tropsch synthesis is a very exothermic reaction, the thermal control of the Fischer-Tropsch synthesis reaction is very important for the stable operation of a device in industrial application. In industrial operation, especially in start-up stage, the device is in a relatively unstable condition with temperature fluctuation, which poses a great challenge to the heat resistance of the catalyst.

In order to improve the activity and stability of the cobalt-based Fischer-Tropsch synthesis catalyst, the active component cobalt is usually loaded on Al₂O₃, SiO₂, TiO₂, ZrO₂ and other carriers. The γ-Al₂O₃ carrier has low hydrothermal stability, and may be undergo hydrothermal reactions in a high hydrothermal atmosphere to convert into AlO(OH). The SiO₂ is not easy to react with the steam, but molded grains are easy to break after long-time contact with the steam, leading to the rapid reduction of catalyst strength. In addition, an interaction between the SiO₂ and the active component cobalt is weak, Co/SiO₂ catalyst is prone to sintering deactivation under large fluctuations of temperature. The hydrothermal stability of TiO₂ is significantly better than that of γ-Al₂O₃ and SiO₂, so that TiO₂ is often used as the carrier of the cobalt-based Fischer-Tropsch synthesis catalyst in industry. The TiO₂, as the catalyst carrier, is usually composed of two crystal phases namely anatase and rutile.

CN1230164A discloses a cobalt-based Fischer-Tropsch synthesis catalyst with a titanium dioxide carrier, a ratio of rutile to anatase in the titanium dioxide is lower than 2:3, surface area is lower than 75 m²/g. A pore volume of the carrier is at least 0.45 ml/g by mercury porosity measurement method.

U.S. Pat. No. 6,130,184A discloses a preparation method for a titanium dioxide-loaded cobalt-based Fischer-Tropsch synthesis catalyst, and the catalyst is prepared by mixing TiO₂ or a titanium raw material with a cobalt source, and then molding, drying and calcinating the mixture to obtain the catalyst.

US20160175821A1 discloses a preparation method for chlorine-containing cobalt-based Fischer-Tropsch and use thereof. The catalyst consists of titanium dioxide, at least 5 wt % of cobalt and 0.1 wt % to 15 wt % of promoters, and the promoters comprise manganese, rhenium, noble metals from Group 8-10 of the Periodic Table or a mixture thereof; the catalyst is impregnated with a solution containing chloride ions; and the impregnated catalyst is heated at a temperature of 100° C. to 500° C. for at least 5 minutes to 2 days. The prepared catalyst contains 0.13 wt % to 10 wt % of element chlorine. The solution containing chloride ions is a solution containing one or more metal chloride salts, hydrochloric acid (HCl), one or more organic chlorine compounds, or a combination thereof.

CN105392558A discloses a preparation method for a chlorine-containing Fischer-Tropsch catalyst, which comprises the following step of: (a) making the following substances in contact with titanium dioxide: cobalt and/or cobalt compounds; one or more promoters, wherein the promoters comprise manganese, rhenium, noble metals of 8^{th} to 10^{th} groups, or a mixture thereof; one or more metal chloride salts, HCl hydrochloride, one or more organic chloride compounds, or a combination thereof; and one or more cocatalysts. After treatment at 70° C. to 350° C., the catalyst contains at least 5 wt % of cobalt, 0.1 wt % to 15 wt % of promoter and 0.15 wt % to 3 wt % of element chlorine based on the total weight of the catalyst. The embodiments of the patent confirm that the addition of Cl improves the C₅+ hydrocarbon selectivity. The inventor found that, during the Fischer-Tropsch synthesis, Cl ions in the catalyst may lose gradually, leading to the activity reduction of the catalyst or even rapid deactivation. Although the above prior art can improve the hydrothermal stability, the C₅+ hydrocarbon selectivity, the activity of the catalyst and the stability of the catalyst still need to be further improved. The selectivity to by-products such as CH₄ and C₂H₆ is still high, while main target products of the Fischer-Tropsch synthesis reaction are liquid and solid hydrocarbons with a high added value, and CH₄ is a by-product to be reduced to the greatest extent.

Therefore, there is a need for Fischer-Tropsch synthesis catalyst with simple synthesis process, high hydrothermal stability and low selectivity to CH₄ and C₂H₆.

### SUMMARY

The present invention aims to overcome the problems of poor hydrothermal stability, easy deactivation, complicated manufacturing processes, high selectivity to by-products such as CH₄ and C₂H₆ of a Fischer-Tropsch synthesis catalyst, and to provide a Fischer-Tropsch synthesis catalyst, a preparation method therefor, and an application thereof.

The inventor of the present invention found through research that a loaded cobalt-based catalyst is prone to deactivation during Fischer-Tropsch synthesis and a deactivation mechanism mainly comprises sintering of active phase metal cobalt of the catalyst, carbon deposition on surface of the catalyst, reaction between cobalt phase and carrier, sulfur poisoning, and so on. The inventor also found that, for a TiO₂-loaded cobalt-based catalyst, carbon deposition and growth of crystal grains of active phase metal cobalt in the catalyst are the main reasons of deactivation of the catalyst.

In the present invention, the inventor found that the stability of a TiO₂ carrier in the catalyst can be significantly improved by introducing Zr promoter into Co/TiO₂. The inventor found that, after the introduction of the Zr-containing promoter and a calcinating treatment, the Zr-containing promoter mainly exists in the form of ZrO₂. The existence of ZrO₂ significantly inhibits the growth of crystal grains of active phase metal cobalt in the catalyst, improving the catalyst stability.

The inventor also found that when a salt solution containing Cl ions, such as cobalt chloride, zirconium oxychloride, titanium chloride, titanium oxychloride or/and manganese chloride, is introduced in a preparation process of the catalyst, a catalyst with excellent stability can be obtained, which is mainly because the carbon deposition on the surface of the catalyst and the coating of the TiO₂ carrier on the metal cobalt may be inhibited by the addition of Cl. The method of introducing Cl ions in the preparation process of the catalyst achieves better stability than the catalyst prepared by a post-impregnation method.

However, because a large amount of steam is generated during the Fischer-Tropsch reaction, the inventor found that the Cl ions may lose gradually with the progress of the reaction, resulting in reduction of activity and heavy hydrocarbon selectivity of the catalyst.

A Cl source is further introduced into the catalyst, thus further obtaining a catalyst with excellent stability. Under co-existence of Cl and Zr, Zr can significantly inhibit the loss of Cl ions, and the catalyst not only has good stability, but also has low selectivity to methane, so that there is a certain synergistic effect between Cl and Zr.

The inventor found that, when a ratio of Cl to Zr is controlled to be appropriate, the stability of the catalyst is exceptionally excellent, and the co-existence of Cl and ZrO₂ can not only significantly inhibit the growth of crystal grains of active phase metal cobalt in the catalyst, but also inhibit the carbon deposition on the surface of the catalyst and the coating of TiO₂ carrier on the metal cobalt, so as to keep high activity and C₅+ hydrocarbon selectivity of the catalyst during the reaction.

In the Fischer-Tropsch synthesis, some catalysts have good stability in conversion efficiency of raw material gases CO and H₂, but the selectivity to target products of liquid and solid hydrocarbons is gradually reduced with the reaction, the selectivity to a by-product methane is gradually increased. When Cl and Zr coexist and the ratio of Cl to Zr is controlled to be appropriate, stabilization of the conversion efficiency of CO and H₂ and stabilization of the target products of liquid and solid hydrocarbons may be achieved simultaneously.

In order to achieve the above objects, in a first aspect, the present invention provides a Fischer-Tropsch synthesis catalyst comprising 10 wt % to 45 wt % of Co, 0.01 wt % to 2.5 wt % of Mn, 0.01 wt % to 1.5 wt % of Cl, 0.5 wt % to 8 wt % of ZrO₂, and 35 wt % to 85 wt % of carrier TiO₂ based on the total weight of the catalyst, wherein, a molar ratio of Cl to Zr is 1:20 to 1:0.1; and
the particle size of cobaltosic oxide in the catalyst ranges from 16 nm to 27 nm.

In a second aspect, the present invention provides a preparation method for the Fischer-Tropsch synthesis catalyst above, which comprises the following steps of:
(1) mixing Ti source and Co source for first kneading to obtain a first mixture;
(2) adding Zr source and optional peptizing agent into the first mixture for second kneading to obtain a second mixture;
(3) adding Mn source, Cl source, optional cocatalyst and optional Co source into the second mixture for third kneading to obtain a matrix catalyst; and
(4) drying and calcinating the matrix catalyst to obtain the Fischer-Tropsch synthesis catalyst;
wherein, amounts of the Co source, the Mn source, the Cl source, the Zr source, the Ti source and the cocatalyst are based on the total weight of the catalyst, the content of Co is 10 wt % to 45 wt %, the content of Mn is 0.01 wt % to 2.5 wt %, the content of Cl is 0.01 wt % to 1.5 wt %, the content of ZrO₂ is 0.5 wt % to 8 wt %, and the content of titanium dioxide is 35 wt % to 85 wt %, and the content of cocatalyst is 0 wt % to 6 wt %, and the molar ratio of Cl to Zr is 1:20 to 1:0.1.

In a third aspect, the present invention provides an application of the Fischer-Tropsch synthesis catalyst in the first aspect of the present invention and/or a Fischer-Tropsch synthesis catalyst prepared by the method in the second aspect of the present invention in a Fischer-Tropsch synthesis.

The Fischer-Tropsch synthesis catalyst provided by the present invention has high activity, excellent stability and low selectivity to methane, and is particularly suitable for fixed bed cobalt-based Fischer-Tropsch synthesis process. In a preferred embodiment, the Fischer-Tropsch synthesis catalyst of the present invention is used in the Fischer-Tropsch synthesis, the activity of the catalyst is not inactivated within 500 hours at a single-pass CO conversion rate of 75% under conditions of 215° C., 2 MPa and space velocity of 3 L/(g_{cat}-h) in a synthesis gas of H₂/CO=2, and meanwhile, CH₄ selectivity of the catalyst is not higher than 6.1% or even less than 5% after stable operation.

### DESCRIPTION OF THE DRAWINGS

FIG. 1 shows a change of a CO conversion rate when a catalyst A1 of the present invention and a catalyst D4 of the prior art are used in Fischer-Tropsch synthesis with extension of reaction time.

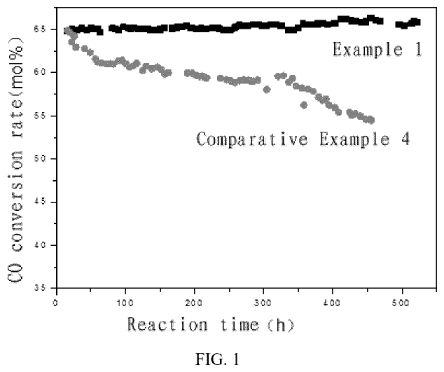

### DESCRIPTION OF EXEMPLARY EMBODIMENTS

Endpoints of ranges and any values disclosed herein are not limited to the accurate ranges or values, and these ranges or values should be understood as comprising values close to these ranges or values. For numerical ranges, endpoint values of the ranges, the endpoint values of the ranges and individual point values, and the individual point values may be combined with each other to obtain one or more new numerical ranges, and these numerical ranges should be regarded as being specifically disclosed herein.

In a first aspect, the present invention provides a Fischer-Tropsch synthesis catalyst, comprising: 10 wt % to 45 wt % of Co, 0.01 wt % to 2.5 wt % of Mn, 0.01 wt % to 1.5 wt % of Cl, 0.5 wt % to 8 wt % of ZrO₂, and 35 wt % to 85 wt % of TiO₂ based on the total weight of the catalyst, a molar ratio of Cl to Zr is 1:20 to 1:0.1; and
a grain size of cobaltosic oxide in the catalyst is 16 nm to 27 nm.

In the catalyst of the present invention, Zr exists in the form of ZrO₂, and Co exists in the form of cobaltosic oxide.

According to the present invention, the titanium dioxide comprises two crystal forms, anatase and rutile, and the content of the anatase is richer than that of rutile in the titanium dioxide.

Further, based on the total amount of the titanium dioxide, the content of the anatase is greater than 50 wt %, and further preferably greater than 60 wt %; and the content of the rutile is less than 40 wt %, and preferably less than 30 wt %.

The content of the titanium dioxide crystal form is determined by an XRD method.

Further, in the catalyst, based on the total weight of the catalyst, the content of Co is 15 wt % to 40% wt %, for example, the content may be 16 wt %, 20 wt %, 30 wt %, 35 wt %, and any value within a range formed by any two values above; the content of Mn is 0.1 wt % to 1.3 wt %, for example, the content may be 0.1 wt %, 0.5 wt %, 1 wt %, 1.3 wt %, and any value within a range formed by any two values above; the content of Cl is 0.08 wt % to 1.2 wt %, preferably 0.09 wt % to 1.1% wt %, for example, the content may be 0.1 wt %, 0.15 wt %, 0.2 wt %, 0.5 wt %, 1.1 wt %, and any value within a range formed by any two values above; the content of ZrO₂ is 0.8 wt % to 6.5 wt %, for example, the content may be 2 wt %, 3 wt %, 5 wt %, 6.5 wt %, and any value within a range formed by any two values above, and the content of titanium dioxide is 45 wt % to 80 wt %, for example, the content may be 48 wt %, 55 wt %, 65 wt %, 70 wt %, 75 wt %, 78 wt %, and any value within a range formed by any two values above, The molar ratio of Cl to Zr is 1:15 to 1:0.2.

Further, the particle size of cobaltosic oxide in the catalyst ranges from 18 nm to 25 nm.

In the catalyst of the present invention, Cl can inhibit carbon deposition on surface of the catalyst. When the content of Cl is less than 0.01 wt %, chloride ions cannot effectively inhibit the carbon deposition; and when the content of Cl is higher than 1.5 wt %, the activity of the catalyst is inhibited. Especially, when the molar ratio of Cl to Zr is 1:20 to 1:0.1, preferably 1:15 to 1:0.2, the catalytic performance and stability of the catalyst are further improved.

In the catalyst of the present invention, there is a synergistic effect between Cl and ZrO₂ in the catalyst, co-existence of Cl and ZrO₂ can not only significantly inhibit growth of crystal grains of active phase metal cobalt in the catalyst, but also inhibit the carbon deposition on the surface of the catalyst, thus maintaining the activity of the catalyst during the reaction. In addition, the catalyst has low selectivity to methane.

Specifically, the catalyst is used in the Fischer-Tropsch synthesis, and before the reaction, a particle size of the metal cobalt in the catalyst is D0; after the catalyst reacts for 20 hours, the particle size of the metal cobalt is D1; and after the catalyst reacts for 500 hours, the particle size of the metal cobalt is D2. When D1−D0/D0≤20%, preferably 0% to 17%; and D2−D0/D0≤35%, preferably 5% to 30%, catalytic activity and stability of the catalyst are further improved, CO conversion rate of the catalyst can reach 53% or more, and selectivity to CH₄ is not higher than 6.1%. Before the catalyst of the present invention is used, a reduction treatment needs to be carried out to form a reduced Fischer-Tropsch synthesis catalyst. In the reduced Fischer-Tropsch synthesis catalyst, Zr exists in the form of ZrO₂, and Co mostly exists in the form of metal Co, and rarely exists in the form of CoO.

Preferably, the Fischer-Tropsch synthesis catalyst of the present invention further comprises a cocatalyst selected from at least one of platinum, ruthenium, rhodium, palladium, yttrium, rhenium, iron, vanadium, aluminum and lanthanum.

The cocatalyst comprised in the present invention can improve the activity, stability and the C₅+ hydrocarbons selectivity.

Further, based on the total weight of the catalyst, a content of the cocatalyst is 0 wt % to 6 wt %, preferably 0.2 wt % to 4 wt %.

In the present invention, after reacting for 500 hours, a retention rate of chloride ions in the catalyst is higher than or equal to 81%.

In a second aspect, the present invention provides a preparation method for the Fischer-Tropsch synthesis catalyst, which comprises the following steps of:
(1) mixing Ti source and Co source for first kneading to obtain a first mixture;
(2) adding Zr source and optional peptizing agent into the first mixture for second kneading to obtain a second mixture;
(3) adding Mn source, Cl source, an optional cocatalyst and an optional Co source into the second mixture for third kneading to obtain a matrix catalyst; and
(4) drying and calcinating the matrix catalyst to obtain the Fischer-Tropsch synthesis catalyst;
wherein, amounts of the Co source, the Mn source, the Cl source, the Zr source, the Ti source and the cocatalyst are based on the total weight of the catalyst, the content of Co is 10 wt % to 45 wt %, the content of Mn is 0.01 wt % to 2.5 wt %, the content of Cl is 0.01 wt % to 1.5 wt %, the content of ZrO₂ is 0.5 wt % to 8 wt %, and the content of titanium dioxide is 35 wt % to 85 wt %, and a content of cocatalyst is 0 wt % to 6 wt %, and the molar ratio of Cl to Zr is 1:20 to 1:0.1.

The inventor of the present invention found by chance that, according to the catalyst prepared by the method of the present invention, ZrO₂ can significantly inhibit the growth of crystal grains of active phase metal cobalt in the catalyst, thus maintaining the stability of the catalyst, and in the catalyst, the retention rate of chloride ions can reach 81% or more.

Preferably, the Co source is selected from at least one of cobalt nitrate, cobalt carbonate, cobalt acetate, cobalt hydroxide and cobalt chloride.

Preferably, the Zr source is selected from at least one of ZrO₂, zirconyl nitrate and zirconium oxychloride.

Preferably, the Mn source is selected from at least one of MnO₂, manganese acetate, manganese nitrate and manganese chloride.

Preferably, the titanium source is selected from at least one of TiO₂, titanium chloride, titanium oxychloride, titanium hydroxide and tetrabutyl titanate.

Preferably, the cocatalyst source is selected from at least one of chloroplatinic acid, ruthenium trichloride, rhodium trichloride, palladium chloride, yttrium nitrate, ammonium perrhenate, ferric nitrate, vanadium oxytrichloride, pseudo-boehmite and lanthanum nitrate.

Preferably, the Cl source is selected from at least one of cobalt chloride, zirconium oxychloride, manganese chloride and hydrochloric acid.

In a preferred embodiment, the Co source and/or the Cl source is cobalt chloride.

In the present invention, when the Co source, the Mn source, the Cl source, the Zr source and the Ti source are all solid substances, in order to facilitate kneading and molding, the peptizing agent is preferably added, and the peptizing agent is used for peptizing added oxides such as TiO₂ and ZrO₂, thus being prone to molding and interacting with other components in the catalyst. Preferably, the peptizing agent is selected from at least one of glacial acetic acid, citric acid, nitric acid, hydrochloric acid, ammonia water and ammonium bicarbonate.

Preferably, the drying is carried out at a temperature of 80° C. to 150° C., and lasts for 2 hours to 48 hours.

Preferably, the calcinating is carried out at a temperature of 300° C. to 650° C., preferably 400° C. to 580° C.; and lasts for 1 hour to 40 hours, preferably 2 hours to 20 hours.

In order to facilitate transportation and further improve the activity of the catalyst, preferably, the reduction treatment is not carried out priorly but the catalyst is reduced in situ before Fischer-Tropsch synthesis, so as to obtain the reduced Fischer-Tropsch synthesis catalyst.

In a preferred specific embodiment, the reduction treatment is carried out at 250° C. to 400° C. in H₂ atmosphere for 5 hours to 100 hours.

The method of the present invention has simple process steps, and the obtained catalyst has low methane selectivity and high activity; more particularly, the catalyst has excellent stability, and is particularly suitable for a fixed bed Fischer-Tropsch synthesis.

In a third aspect, the present invention provides an application of the Fischer-Tropsch synthesis catalyst in the first aspect of the present invention and/or a Fischer-Tropsch synthesis catalyst prepared by the method in the second aspect of the present invention in a Fischer-Tropsch synthesis reaction.

According to the present invention, the particle size of metal cobalt in the catalyst before the reaction is D0; after reacting for 20 hours, the particle size of the metal cobalt in the catalyst is D1; and after reacting for 500 hours, the particle size of the metal cobalt in the catalyst is D2. (D1−D0)/D0×100%≤20%; and (D2−D0)/D0×100%≤35%.

Further, (D1−D0)/D0×100% is 0% to 17%; and (D2−D0)/D0×100% is 5% to 30%.

The present invention will be described in detail herein after with reference to the examples.

Tests involved in examples and comparative examples are as follows:
contents of various components in the catalyst are determined by XRF;
in the catalyst, the molar ratio of Cl to Zr is determined by XRF;
in the catalyst, the particle size of the metal cobalt is calculated through a Scherrer formula of XRD; and
the content of titanium dioxide powder and contents of the anatase crystal form in the catalyst before and after the reaction are determined by XRD.

### Example 1

200 g of TiO₂ powder (with a specific surface area of 30 m²/g to 70 m²/g, wherein the content of anatase was 90 wt %) and 30.4 g of cobalt hydroxide were put into a kneader for first kneading for 30 minutes. 21.7 g of zirconyl nitrate (ZrO(NO₃)₂·2H₂O) and 15 g of glacial acetic acid were dissolved in 15 ml of deionized water and then added into the kneader for second kneading for 30 minutes. 3.3 g of manganese nitrate (Mn(NO₃)₂), 96.5 g of cobalt nitrate (Co(NO₃)₂·6H₂O) and 1.5 g of anhydrous cobalt chloride (CoCl₂) were dissolved in 85 g of deionized water and then added into the kneader for third kneading for 60 minutes. After being evenly mixed in the kneader, the mixtures were shaped using extrusion, dried at 120° C. for 10 hours, and then calcinated at 550° C. for 3 hours to obtain a catalyst A1.

According to an XRF test, based on a total weight of the catalyst A1, a content of Co was 15 wt %, a content of Mn was 0.4 wt %, a content of Cl was 0.3 wt %, a content of ZrO₂ was 4.3 wt %, and a content of titanium dioxide was 74.9 wt % (in the titanium dioxide, the content of anatase was 72 wt %, the content of rutile was 28 wt %).

### Example 2

200 g of TiO₂ powder (the same as that in Example 1) and 91.2 g of cobalt hydroxide were put into a kneader for first kneading for 30 minutes. 43.4 g of zirconyl nitrate (ZrO(NO₃)₂·2H₂O) and 25 g of glacial acetic acid were dissolved in 30 g of deionized water and then added into the kneader for second kneading for 30 minutes. 13.2 g of manganese nitrate (Mn(NO₃)₂), 96.5 g of cobalt nitrate (Co(NO₃)₂·6H₂O) and 4.5 g of anhydrous cobalt chloride (CoCl₂) were dissolved in 70 g of deionized water and then added into the kneader for third kneading for 60 minutes. After being evenly mixed in the kneader, the mixtures were shaped using extrusion, dried at 140° C. for 18 hours, and then calcinated at 420° C. for 10 hours to obtain a catalyst A2.

According to an XRF test, based on a total weight of the catalyst A2, a content of Co was 24 wt %, a content of Mn was 1.2 wt %, a content of Cl was 0.6 wt %, a content of ZrO₂ was 7 wt %, and a content of titanium dioxide was 60 wt % (in the titanium dioxide, the content of anatase was 80 wt %, the content of rutile was 20 wt %).

### Example 3

200 g of TiO₂ powder (the same as that in Example 1) and 162.4 g of cobalt hydroxide were put into a kneader for first kneading for 30 minutes. 43.4 g of zirconyl nitrate (ZrO(NO₃)₂·2H₂O) and 15 g of glacial acetic acid were dissolved in 50 g of deionized water and then added into the kneader for second kneading for 30 minutes. 22 g of manganese nitrate (Mn(NO₃)₂), 193 g of cobalt nitrate (Co(NO₃)₂·6H₂O) and 13 g of anhydrous cobalt chloride (CoCl₂) were dissolved in 90 ml of deionized water and then added into the kneader for third kneading for 60 minutes. After being evenly mixed in the kneader, the mixtures were shaped using extrusion, dried at 90° C. for 5 hours, and then calcinated at 500° C. for 10 hours to obtain a catalyst A3.

According to an XRF test, based on a total weight of the catalyst A3, a content of Co was 33 wt %, a content of Mn was 1.5 wt %, a content of Cl was 1.1 wt %, a content of ZrO₂ was 5.2 wt %, and a content of titanium dioxide was 45 wt % (in the titanium dioxide, the content of anatase was 75 wt %, the content of rutile was 25 wt %).

### Example 4

200 g of TiO₂ powder (the same as that in Example 1) and 236.7 g of cobalt hydroxide were put into a kneader for kneading for 30 minutes. 21.7 g of zirconyl nitrate (ZrO(NO₃)₂·2H₂O) and 25 g of glacial acetic acid were dissolved in 15 g of deionized water and then added into the kneader for continuous kneading for 30 minutes. 27.5 g of manganese nitrate (Mn(NO₃)₂), 96.5 g of cobalt nitrate (Co(NO₃)₂·6H₂O) and 20 g of anhydrous cobalt chloride (CoCl₂) were dissolved in 85 g of deionized water and then added into the kneader for continuous kneading. After being evenly mixed in the kneader, the mixtures were shaped using extrusion, dried at 100° C. for 12 hours, and then calcinated at 600° C. for 2 hours to obtain a catalyst A4.

According to an XRF test, based on a total weight of the catalyst A4, a content of Co was 38 wt %, a content of Mn was 1.8 wt %, a content of Cl was 1.4 wt %, a content of ZrO₂ was 2 wt %, and a content of titanium dioxide was 43 wt % (in the titanium dioxide, the content of anatase was 64 wt %, the content of rutile was 36 wt %).

### Example 5

246 g of TiO₂ powder (the same as that in Example 1) and 122 g of cobalt hydroxide were put into a kneader for kneading for 30 minutes. 13.9 g of ZrO₂ and 5 g of concentrated hydrochloric acid were dissolved in 50 g of deionized water and then added into the kneader for continuous kneading for 30 minutes. 12.8 g of MnO₂ and 8 g of titanium oxychloride (TiOCl₂·8H₂O) were dissolved in 65 g of deionized water and then added into the kneader for continuous kneading. After being evenly mixed in the kneader, the mixtures were shaped using extrusion, dried at 80° C. for 24 hours, and then calcinated at 360° C. for 7 hours to obtain a catalyst A5.

According to an XRF test, based on a total weight of the catalyst A5, a content of Co was 20 wt %, a content of Mn was 2 wt %, a content of Cl was 0.08 wt %, a content of ZrO₂ was 4 wt %, and a content of titanium dioxide was 65 wt % (in the titanium dioxide, the content of anatase was 85 wt %, the content of rutile was 15 wt %).

### Example 6

A Fischer-Tropsch synthesis catalyst was prepared according to the method in Example 1, with a difference that an amount of anhydrous cobalt chloride (CoCl₂) was 0.8 g, and the rest were the same as those in Example 1, so that a catalyst A6 was finally obtained.

According to an XRF test, based on a total weight of the catalyst A6, a content of Co was 14.8 wt %, a content of Mn was 0.41 wt %, a content of Cl was 0.15 wt %, a content of ZrO₂ was 4.4 wt %, and a content of titanium dioxide was 75.3 wt % (in the titanium dioxide, the content of anatase was 72 wt %, the content of rutile was 28 wt %).

### Example 7

A Fischer-Tropsch synthesis catalyst was prepared according to the method in Example 1, with a difference that an amount of anhydrous cobalt chloride (CoCl₂) was 10 g, and the rest were the same as those in Example 1, so that a catalyst A7 was finally obtained.

According to an XRF test, based on a total weight of the catalyst A7, a content of Co was 17 wt %, a content of Mn was 0.4 wt %, a content of Cl was 1.3 wt %, a content of ZrO₂ was 4 wt %, and a content of titanium dioxide was 75 wt % (in the titanium dioxide, the content of anatase was 72 wt %, the content of rutile was 28 wt %).

### Example 8

A Fischer-Tropsch synthesis catalyst was prepared according to the method in Example 1, with a difference that a calcinating temperature was 650° C., and the rest were the same as those in Example 1, so that a catalyst A8 was finally obtained.

According to an XRF test, based on a total weight of the catalyst A8, a content of Co was 15 wt %, a content of Mn was 0.4 wt %, a content of Cl was 0.12 wt %, a content of ZrO₂ was 4.3 wt %, and a content of titanium dioxide was 74.9 wt % (in the titanium dioxide, the content of anatase was 55 wt %, the content of rutile was 45 wt %).

### Example 9

A Fischer-Tropsch synthesis catalyst was prepared according to the method in Example 1, with a difference that a content of titanium dioxide in a crystal form namely anatase in TiO₂ powder used was 60 wt %, and the rest were the same as those in Example 1, so that a catalyst A9 was finally obtained.

According to an XRF test, based on a total weight of the catalyst A9, a content of Co was 15 wt %, a content of Mn was 0.4 wt %, a content of Cl was 0.3 wt %, a content of ZrO₂ was 4.3 wt %, and a content of titanium dioxide was 74.9 wt % (in the titanium dioxide, the content of anatase was 51 wt %, the content of rutile was 49 wt %).

### Example 10

A Fischer-Tropsch synthesis catalyst was prepared according to the method in Example 1, with a difference that 45 g of acidic silica sol containing 20 wt % of SiO₂ was added, and the rest were the same as those in Example 1, so that a catalyst A10 was finally obtained.

According to an XRF test, based on a total weight of the catalyst A9, a content of Co was 14 wt %, a content of Mn was 3.6 wt %, a content of Cl was 0.3 wt %, a content of ZrO₂ was 4 wt %, a content of SiO₂ was 3.5 wt %, and a content of titanium dioxide was 73 wt % (in the titanium dioxide, the content of anatase was 72 wt %, the content of rutile was 28 wt %).

### Comparative Example 1

44 g of Co(NO₃)₂·6H₂O was dissolved in 15 g of deionized water and stirred to prepare a solution, and 100 g of dried TiO₂ carrier (the content of anatase was 100 wt %) was added into the above solution, dried and dehydrated at 85° C. for 4 hours, and then heated to 120° C. for drying for 10 hours. 36.9 g of Co(NO₃)₂·6H₂O was dissolved in 15 g of deionized water to prepare a solution, and a dried sample was added into the solution, dried and dehydrated at 85° C. for 4 hours again, and then heated to 120° C. for drying for 10 hours. Subsequently, the dried mixture was heated to 250° C. in a rate of 1° C./min for calcinating for 4 hours to prepare a catalyst D1.

According to an XRF test, based on a total weight of the catalyst D1, a content of Co was 14.2 wt % and a content of titanium dioxide was 81.3 wt % (in the titanium dioxide, the content of anatase was 94 wt %, the content of rutile was 6 wt %).

### Comparative Example 2

TiO₂ in Comparative Example 1 was replaced with ZrO₂, and the rest were the same as those in Comparative Example 1, so that a catalyst D2 was finally obtained.

According to an XRF test, based on a total weight of the catalyst D2, a content of Co was 14.5 wt % and a content of ZrO₂ was 81 wt %.

### Comparative Example 3

A catalyst was prepared according to the method in Example 1, with differences that zirconyl nitrate was not used and a calcinating temperature of the catalyst was 500° C., and the rest were the same as those in Example 1, so that a catalyst D3 was finally obtained.

According to an XRF test, based on a total weight of the catalyst D3, a content of Co was 15.5 wt %, a content of Mn was 0.5 wt %, a content of Cl was 0.3 wt %, and a content of titanium dioxide was 76 wt % (in the titanium dioxide, the content of anatase was 65 wt %, the content of rutile was 35 wt %).

### Comparative Example 4

A catalyst was prepared according to the method in Example 1, with a difference that 1.5 g of anhydrous cobalt chloride was replaced with 6 g of cobalt acetate, and the rest were the same as those in Example 1, so that a catalyst D4 was finally obtained.

According to an XRF test, based on a total weight of the catalyst D4, a content of Co was 15.5 wt %, a content of Mn was 0.39 wt %, a content of ZrO₂ was 3.9 wt %, and a content of titanium dioxide was 74.6 wt % (in the titanium dioxide, the content of anatase was 72 wt %, the content of rutile was 28 wt %).

### Comparative Example 5

A Fischer-Tropsch synthesis catalyst was prepared according to the method in Example 1, with a difference that an amount of anhydrous cobalt chloride (CoCl₂) was 12 g, and the rest were the same as those in Example 1, so that a catalyst D5 was finally obtained.

According to an XRF test, based on a total weight of the catalyst D5, a content of Co was 16 wt %, a content of Mn was 0.4 wt %, a content of Cl was 2 wt %, a content of ZrO₂ was 4.1 wt %, and a content of titanium dioxide was 74 wt % (in the titanium dioxide, the content of anatase was 72 wt %, the content of rutile was 28 wt %).

### Comparative Example 6

A Fischer-Tropsch synthesis catalyst was prepared according to the method in Example 1, with a difference that an amount of zirconyl nitrate was 1.7 g, and the rest were the same as those in Example 1, so that a catalyst D6 was finally obtained.

According to an XRF test, based on a total weight of the catalyst D6, a content of Co was 15.4 wt %, a content of Mn was 0.44 wt %, a content of Cl was 0.31 wt %, a content of ZrO₂ was 0.3 wt %, and a content of titanium dioxide was 78.4 wt % (the content of anatase was 70 wt %, the content of rutile was 30 wt %).

### Comparative Example 7

A Fischer-Tropsch synthesis catalyst was prepared according to the method in Example 1, with a difference that an amount of zirconyl nitrate was 68 g, and the rest were the same as those in Example 1, so that a catalyst D7 was finally obtained.

According to an XRF test, based on a total weight of the catalyst D7, a content of Co was 13.5 wt %, a content of Mn was 0.36 wt %, a content of Cl was 0.33 wt %, a content of ZrO₂ was 10.9 wt %, and a content of titanium dioxide was 70 wt % (in the titanium dioxide, the content of anatase was 76 wt %, the content of rutile was 24 wt %).

### Comparative Example 8

A Fischer-Tropsch synthesis catalyst was prepared according to the method in Example 1, with a difference that 21.7 g of zirconyl nitrate was replaced with 9 g of magnesium oxide, and the rest were the same as those in Example 1, so that a catalyst D8 was finally obtained.

According to an XRF test, based on a total weight of the catalyst D8, a content of Co was 15.1 wt %, a content of Mn was 0.41 wt %, a content of Cl was 0.22 wt %, a content MgO was 3.9 wt %, and a content of titanium dioxide was 75 wt % (in the titanium dioxide, the content of anatase was 65 wt %, the content of rutile was 35 wt %).

### Comparative Example 9

A Fischer-Tropsch synthesis catalyst was prepared according to the method in Example 1, with a difference that 21.7 g of zirconyl nitrate was replaced with 9 g of cerium dioxide, and the rest were the same as those in Example 1, so that a catalyst D9 was finally obtained.

According to an XRF test, based on a total weight of the catalyst D8, a content of Co was 15.1 wt %, a content of Mn was 0.41 wt %, a content of Cl was 0.21 wt %, a content of CeO₂ was 3.9 wt %, and a content of titanium dioxide was 75 wt % (in the titanium dioxide, the content of anatase was 67 wt %, the content of rutile was 33 wt %).

### Comparative Example 10

A Fischer-Tropsch synthesis catalyst was prepared according to the method in Example 1, with differences that a content of zirconyl nitrate was adjusted to be 2 g and 15 g of glacial acetic acid was replaced with 15 g of concentrated hydrochloric acid, and the rest were the same as those in Example 1, so that a catalyst D10 was finally obtained.

According to an XRF test, based on a total weight of the catalyst D10, a content of Co was 16 wt %, a content of Mn was 0.4 wt %, a content of Cl was 1.2 wt %, a content of ZrO₂ was 0.1 wt %, and a content of titanium dioxide was 75.8 wt % (the content of anatase was 65 wt %, the content of rutile was 35 wt %).

### Comparative Example 11

A Fischer-Tropsch synthesis catalyst was prepared according to the method in Example 1, with differences that 21.7 g of zirconyl nitrate was replaced with 35 g of ZrO₂ and 1.5 g of anhydrous cobalt chloride (CoCl₂) was adjusted to be 0.5 g, and the rest were the same as those in Example 1, so that a catalyst D11 was finally obtained.

According to an XRF test, based on a total weight of the catalyst D11, a content of Co was 13 wt %, a content of Mn was 0.4 wt %, a content of Cl was 0.1 wt %, a content of ZrO₂ was 12 wt %, and a content of titanium dioxide was 70 wt % (in the titanium dioxide, the content of anatase was 81 wt, the content of rutile was 19 wt %).

### Comparative Example 12

200 g of TiO₂ powder (with a specific surface area of 30 m²/g to 70 m²/g, wherein a content of a crystal form namely anatase was 90 wt %) and 30.4 g of cobalt hydroxide were put into a kneader. 3.3 g of manganese nitrate (Mn(NO₃) 2), 21.7 g of zirconyl nitrate (ZrO(NO₃)₂·2H₂O), 96.5 g of cobalt nitrate (Co(NO₃)₂·6H₂O), 1.5 g of anhydrous cobalt chloride (CoCl₂) and 15 g of glacial acetic acid were dissolved in 85 g of deionized water, and then added into the kneader for kneading for 60 minutes. After being evenly mixed in the kneader, the mixtures were shaped using extrusion, dried at 120° C. for 10 hours, and then calcinated at 550° C. for 3 hours to obtain a catalyst D12.

According to an XRF test, based on a total weight of the catalyst D12, a content of Co was 15 wt %, a content of Mn was 0.4 wt %, a content of Cl was 0.3 wt %, a content of ZrO₂ was 4.3 wt %, and a content of titanium dioxide was 74.9 wt % (in the titanium dioxide, the content of anatase was 72 wt %, the content of rutile was 28 wt %).

Molar ratios of Cl to Zr, particle sizes of Co₃O₄ and crystal forms of titanium dioxide in each catalyst in examples and comparative examples were shown in Table 1.

TABLE 1

| | | Particle size | Content of anatase |
|---|---|---|---|
| | Molar ratio | of Co₃O₄ | crystal form |
| Catalyst | of Cl to Zr | (nm) | (wt %) |
| A1 | 1:4.1 | 20 | 72 |
| A2 | 1:3.4 | 21 | 80 |
| A3 | 1:1.4 | 23 | 75 |
| A4 | 1:0.4 | 25 | 64 |
| A5 | 1:14.4 | 17 | 85 |
| A6 | 1:8.5 | 21 | 72 |
| A7 | 1:0.9 | 19 | 72 |
| A8 | 1:10.3 | 26 | 55 |
| A9 | 1:4.1 | 23 | 51 |
| A10 | 1:3.8 | 19 | 72 |
| D1 | — | 23 | 94 |
| D2 | — | 19 | — |
| D3 | — | 29 | 65 |
| D4 | — | 23 | 72 |
| D5 | 1:0.5 | 19 | 72 |
| D6 | 1:0.28 | 25 | 70 |
| D7 | 1:9.5 | 18 | 76 |
| D8 | — | 32 | 65 |
| D9 | — | 17 | 67 |
| D10 | 1:0.02 | 26 | 65 |
| D11 | 1:34.6 | 18 | 81 |
| D12 | 1:4.1 | 28 | 72 |

### Performance Test of Catalysts

The 0.5 g catalysts A1 to A9 and D1 to D10 were respectively filled into a 10 mL fixed bed reactor. The catalysts were activated in a fixed bed reactor at 350° C. in a H₂ atmosphere for 20 hours first, and then cooled to 180° C. after finishing reduction. A reaction gas was provided, and a reaction temperature was reached by heating to evaluate performances of the catalysts.

The performances of the catalysts were evaluated at 215° C. and 2 MPa in a syngas of H₂/CO (molar ratio)=2 under a space velocity of 3 L/(g_{cat}-h).

Structural parameters of the catalysts in different reaction stages were shown in Table 2. Catalytic performances of the catalysts were shown in Table 3.

TABLE 2

| | | | | | | Retention rate |
|---|---|---|---|---|---|---|
| | | | | | | of chloride ions |
| | | | (D1 − | | (D2 − | in catalyst 500 |
| | D0 | D1 | D0)/D0 × | D2 | D0)/D0 × | hours later |
| Catalyst | (nm) | (nm) | 100% | (nm) | 100% | (%) |
| A1 | 20 | 21 | 5 | 22 | 10 | 89 |
| A2 | 20 | 22 | 10 | 22 | 10 | 87 |
| A3 | 22 | 23 | 5 | 23 | 5 | 89 |
| A4 | 24 | 25 | 4 | 27 | 13 | 81 |
| A5 | 18 | 21 | 17 | 23 | 28 | 86 |
| A6 | 21 | 21 | 0 | 23 | 10 | 85 |
| A7 | 20 | 22 | 10 | 23 | 15 | 88 |
| A8 | 24 | 24 | 0 | 26 | 8 | 92 |
| A9 | 22 | 23 | 5 | 26 | 18 | 88 |
| A10 | 19 | 20 | 5 | 22 | 16 | 90 |
| D1 | 20 | 23 | 15 | 35 | 75 | — |
| D2 | 17 | 19 | 12 | 36 | 112 | — |
| D3 | 27 | 30 | 11 | 35 | 30 | 75 |
| D4 | 23 | 26 | 13 | 28 | 22 | — |
| D5 | 19 | 24 | 26 | 30 | 58 | 82 |
| D6 | 24 | 28 | 17 | 34 | 42 | 79 |
| D7 | 19 | 23 | 21 | 29 | 53 | 91 |
| D8 | 28 | 32 | 14 | 34 | 21 | 70 |
| D9 | 17 | 19 | 12 | 32 | 88 | 76 |
| D10 | 25 | 26 | 4 | 35 | 40 | 74 |
| D11 | 18 | 18 | 0 | 26 | 44 | 88 |
| D12 | 27 | 29 | 7 | 32 | 19 | 82 |

TABLE 3

| | Reaction for 20 hours | | Reaction for 500 hours | |
|---|---|---|---|---|
| | CO conversion | CH₄ | CO conversion | tCH₄ |
| | rate | Selectivity | rate | Selectivity |
| Catalyst | (%) | (wt %) | (%) | (wt %) |
| A1 | 66 | 4.7 | 68 | 4.6 |
| A2 | 56 | 4.8 | 59 | 5 |
| A3 | 75 | 4.6 | 75 | 4.5 |
| A4 | 58 | 5.2 | 59 | 5.4 |
| A5 | 58 | 5.6 | 56 | 5.5 |
| A6 | 62 | 5.5 | 63 | 5.3 |
| A7 | 67 | 5.3 | 70 | 4.5 |
| A8 | 54 | 5.9 | 54 | 6.1 |
| A9 | 55 | 5.8 | 53 | 5.9 |
| A10 | 62 | 5 | 63 | 4.2 |
| DI | 36 | 7.1 | 33 | 8.2 |
| D2 | 20 | 12.5 | 14 | 14.6 |
| D3 | 56 | 7 | 50 | 8.2 |
| D4 | 64 | 6.4 | 53 | 7.8 |
| D5 | 39 | 6.5 | 42 | 6 |
| D6 | 48 | 7 | 43 | 7.8 |
| D7 | 32 | 8 | 25 | 8.9 |
| D8 | 58 | 6.5 | 46 | 6.8 |
| D9 | 35 | 7.9 | 28 | 8.5 |
| D10 | 48 | 7 | 20 | 8.2 |
| D11 | 51 | 6.5 | 35 | 6.9 |
| D12 | 59 | 5 | 58 | 6.5 |

By comparing the catalysts prepared in Examples 1 to 10 and Comparative Examples 1 to 12 according to the results in Tables 1 to 3, it can be seen that the catalyst prepared by the method of the present invention satisfies that the content of Co is 10 wt % to 45 wt %, the content of Mn is 0.01 wt % to 2.5 wt %, the content of Cl is 0.01 wt % to 1.5 wt %, the content of ZrO₂ is 0.5 wt % to 8 wt %, and the content of carrier TiO₂ is 35 wt % to 85 wt %, and when the molar ratio of Cl to Zr is 1:20 to 1:0.1, after reacting for 20 hours, a growth rate of the particle size of the metal cobalt is not higher than 17%, the CO conversion rate can reach to 54% or more, and the selectivity to CH₄ is 5.9% or less. After reacting for 500 hours, the growth rate of the particle size of the metal cobalt is not higher than 28%, the retention rate of chloride ions in the catalyst is 81% or more at the moment, the CO conversion rate is 53% or more, and the selectivity to CH₄ is 6.1% or less, so that good catalytic activity and catalyst stability are achieved.

The catalyst of Comparative example 12 is relatively stable, but the methane selectivity gradually increased.

Those described above are preferred embodiments of the present invention, but are not intended to limit the present invention. Within the scope of the technical concept of the present invention, many simple modifications can be made to the technical solutions of the present invention, comprising the combination of various technical features in any other suitable way. These simple modifications and combinations shall also be regarded as the contents disclosed by the present invention and belong to the protection scope of the present invention.

1. A Fischer-Tropsch synthesis catalyst, wherein, based on a total weight of the catalyst, the Fischer-Tropsch synthesis catalyst comprises: 10 wt % to 45 wt % of Co, 0.01 wt % to 2.5 wt % of Mn, 0.01 wt % to 1.5 wt % of Cl, 0.5 wt % to 8 wt % of ZrO₂, and 35 wt % to 85 wt % of carrier TiO₂; wherein, a molar ratio of Cl to Zr is 1:20 to 1:0.1; and
a particle size of cobaltosic oxide in the catalyst ranges from 16 nm to 27 nm.

2. The catalyst according to claim 1, wherein the titanium dioxide comprises anatase and rutile crystal forms, and the content of anatase is richer than that of rutile in the titanium dioxide.

3. The catalyst according to claim 1- or 2, wherein, based on a total amount of the titanium dioxide, the content of the anatase is greater than 50 wt %, and the content of the rutile is less than 40 wt %; and
preferably, based on the total amount of the titanium dioxide, the content of the anatase is greater than 60 wt %, and the content of the rutile is less than 30 wt %.

4. The catalyst according to claim 1, wherein, based on the total weight of the catalyst, the Fischer-Tropsch synthesis catalyst comprises: 15 wt % to 40 wt % of Co, 0.1 wt % to 1.3 wt % of Mn, 0.08 wt % to 1.2 wt % of Cl, 0.8 wt % to 6.5 wt % of ZrO₂, and 45 wt % to 80 wt % of titanium dioxide; wherein, a molar ratio of Cl to Zr is 1:15 to 1:0.2; and
the particle size of cobaltosic oxide in the catalyst ranges from 18 nm to 25 nm.

5. The catalyst according to claim 1, wherein the catalyst further comprises a cocatalyst selected from at least one of platinum, ruthenium, rhodium, palladium, yttrium, rhenium, iron, vanadium, silicon, aluminum and lanthanum; and
preferably, based on the total weight of the catalyst, a content of the cocatalyst is 0 wt % to 6 wt %.

6. A preparation method for the Fischer-Tropsch synthesis catalyst according to claim 1, comprising the following steps of:
(1) mixing Ti source and Co source for first kneading to obtain a first mixture;
(2) adding Zr source and optional peptizing agent into the first mixture for second kneading to obtain a second mixture;
(3) adding Mn source, Cl source, optional cocatalyst and optional Co source into the second mixture for third kneading to obtain a matrix catalyst; and
(4) drying and calcinating the matrix catalyst to obtain the Fischer-Tropsch synthesis catalyst;
wherein, amounts of the Co source, the Mn source, the Cl source, the Zr source, the Ti source and the cocatalyst are based on the total weight of the catalyst, the content of Co is 10 wt % to 45 wt %, the content of Mn is 0.01 wt % to 2.5 wt %, the content of Cl is 0.01 wt % to 1.5 wt %, the content of ZrO₂ is 0.5 wt % to 8 wt %, and the content of titanium dioxide is 35 wt % to 85 wt %, and a content of cocatalyst is 0 wt % to 6 wt %, and the molar ratio of Cl to Zr is 1:20 to 1:0.1.

7. The method according to claim 6, wherein the Co source is selected from at least one of cobalt nitrate, cobalt carbonate, cobalt acetate, cobalt hydroxide and cobalt chloride;
preferably, the Zr source is selected from at least one of ZrO₂, zirconyl nitrate and zirconium oxychloride;
preferably, the Mn source is selected from at least one of MnO₂, manganese acetate, manganese nitrate and manganese chloride;
preferably, the Ti source is selected from at least one of TiO₂, titanium chloride, titanium oxychloride, titanium hydroxide and tetrabutyl titanate; and
preferably, the cocatalyst source is selected from at least one of chloroplatinic acid, ruthenium trichloride, rhodium trichloride, palladium chloride, yttrium nitrate, ammonium perrhenate, ferric nitrate, vanadium oxytrichloride, silica sol, pseudo-boehmite and lanthanum nitrate.

8. The method according to claim 6, wherein the peptizing agent is selected from at least one of glacial acetic acid, citric acid, nitric acid, hydrochloric acid, ammonia water and ammonium bicarbonate.

9. The method according to claim 6, wherein the Cl source is selected from at least one of cobalt chloride, zirconium oxychloride, manganese chloride and hydrochloric acid.

10. The method according to claim 6, wherein the first kneading lasts for 12 minutes to 120 minutes, the second kneading lasts for 12 minutes to 120 minutes, and the third kneading lasts for 12 minutes to 120 minutes;
preferably, the drying is carried out at a temperature of 80° C. to 150° C., and lasts for 2 hours to 48 hours; and
preferably, the calcinating is carried out at a temperature of 300° C. to 650° C., and lasts for 1 hour to 40 hours.

11. A method of using the Fischer-Tropsch synthesis catalyst according to claim 1 in a Fischer-Tropsch synthesis.

12. The method according to claim 11, wherein the particle size of metal cobalt in the catalyst before the reaction is D0; after reacting for 20 hours, the particle size of the metal cobalt in the catalyst is D1; and after reacting for 500 hours, the particle size of the metal cobalt in the catalyst is D2;
(D1−D0)/D0×100%≤20%; (D2−D0)/D0×100%≤35%; and
preferably, (D1−D0)/D0×100% is 0% to 17%; (D2−D0)/D0×100% is 5% to 30%.

13. The catalyst according to claim 2, wherein, based on a total amount of the titanium dioxide, the content of the anatase is greater than 50 wt %, and the content of the rutile is less than 40 wt %; and
preferably, based on the total amount of the titanium dioxide, the content of the anatase is greater than 60 wt %, and the content of the rutile is less than 30 wt %.

14. The catalyst according to claim 2, wherein, based on the total weight of the catalyst, the Fischer-Tropsch synthesis catalyst comprises: 15 wt % to 40 wt % of Co, 0.1 wt % to 1.3 wt % of Mn, 0.08 wt % to 1.2 wt % of Cl, 0.8 wt % to 6.5 wt % of ZrO₂, and 45 wt % to 80 wt % of titanium dioxide; wherein, a molar ratio of Cl to Zr is 1:15 to 1:0.2; and
the particle size of cobaltosic oxide in the catalyst ranges from 18 nm to 25 nm.

15. The catalyst according to claim 3, wherein, based on the total weight of the catalyst, the Fischer-Tropsch synthesis catalyst comprises: 15 wt % to 40 wt % of Co, 0.1 wt % to 1.3 wt % of Mn, 0.08 wt % to 1.2 wt % of Cl, 0.8 wt % to 6.5 wt % of ZrO₂, and 45 wt % to 80 wt % of titanium dioxide; wherein, a molar ratio of Cl to Zr is 1:15 to 1:0.2; and
the particle size of cobaltosic oxide in the catalyst ranges from 18 nm to 25 nm.

16. The catalyst according to claim 2, wherein the catalyst further comprises a cocatalyst selected from at least one of platinum, ruthenium, rhodium, palladium, yttrium, rhenium, iron, vanadium, silicon, aluminum and lanthanum; and
preferably, based on the total weight of the catalyst, a content of the cocatalyst is 0 wt % to 6 wt %.

17. The catalyst according to claim 3, wherein the catalyst further comprises a cocatalyst selected from at least one of platinum, ruthenium, rhodium, palladium, yttrium, rhenium, iron, vanadium, silicon, aluminum and lanthanum; and
preferably, based on the total weight of the catalyst, a content of the cocatalyst is 0 wt % to 6 wt %.

18. The catalyst according to claim 4, wherein the catalyst further comprises a cocatalyst selected from at least one of platinum, ruthenium, rhodium, palladium, yttrium, rhenium, iron, vanadium, silicon, aluminum and lanthanum; and
preferably, based on the total weight of the catalyst, a content of the cocatalyst is 0 wt % to 6 wt %.

19. The method according to claim 7, wherein the peptizing agent is selected from at least one of glacial acetic acid, citric acid, nitric acid, hydrochloric acid, ammonia water and ammonium bicarbonate.

20. The method according to claim 7, wherein the Cl source is selected from at least one of cobalt chloride, zirconium oxychloride, manganese, chloride and hydrochloric acid.
